(19) United States
(12) Patent Application Publication
(10) Pub. No.: US 2013/0303401 A1
(43) Pub. Date: Nov. 14, 2013

(54) SYSTEMS AND METHODS FOR DIAGNOSING RENAL CELL CARCINOMA

(71) Applicants: Warren Kruger, Rydal, PA (US); Alaaeldin Mustafa, Philadelphia, PA (US)

(72) Inventors: Warren Kruger, Rydal, PA (US); Alaaeldin Mustafa, Philadelphia, PA (US)

(21) Appl. No.: 13/979,745

(22) PCT Filed: Jan. 13, 2012
(86) PCT No.: PCT/US12/21228

§ 371 Date: Jul. 15, 2013

Related U.S. Application Data
(60) Provisional application No. 61/432,284, filed on Jan. 13, 2011.

(51) Int. Cl. G01N 33/68; G06F 19/00

(52) U.S. Cl. CPC G01N 33/6806; G06F 19/34
USPC 506/12; 702/21

(57) ABSTRACT

Systems, methods, and computer readable media for diagnosing or characterizing kidney cancer based on serum amino acid profiles are provided. Serum amino acid concentrations, and optionally also serum creatinine concentration, are determined in serum obtained from a subject and compared against reference concentration profiles. The condition or prognosis of the subject may be determined based on comparisons of patient samples with reference profiles.

### CROSS REFERENCE TO RELATED APPLICATIONS

This application claims priority to U.S. Provisional Application No. 61/432,284 filed on Jan. 13, 2011, the entire contents of which are incorporated by reference herein, in their entirety and for all purposes.

### FIELD OF THE INVENTION

The invention relates generally to the field of cancer diagnostics. More particularly, the invention relates to systems and methods for diagnosing kidney cancer and determining the prognosis of kidney cancer patients.

### BACKGROUND OF THE INVENTION

Various publications, including patents, published applications, technical articles and scholarly articles are cited throughout the specification. Each of these cited publications is incorporated by reference herein, in its entirety and for all purposes.

In the United States, it is estimated that there will have been over 50,000 new cases of Renal Cell Carcinoma (RCC) diagnosed in 2010, and more than 13,000 deaths from the disease. Men are 1.5 times more likely to develop kidney cancer compared to women, and kidney cancer is the eighth leading cause of cancer death in men and the fourteenth in women. The most common subtypes of RCC are clear cell carcinomas, accounting for about 70% of the disease, followed by the papillary form that accounts for about 20% of the patients.

Prognosis in RCC is very much dependent on the stage at which the disease is caught. Small tumors confined to the kidney have 5-year survival rates as high as 90%, while advanced tumors that have metastasized outside the kidney have rates less than 20%. Unfortunately, most individuals with locally confined disease have no obvious symptoms, and therefore, about half of the individuals with the disease are detected late. In fact, most early stage kidney cancer is detected serendipitously, usually when a patient is having an abdominal CT scan for some other condition. Given the large differences in outcome between early and late stage tumors, a blood-based screening test to detect individuals with early stage tumors would be extremely valuable.

### SUMMARY OF THE INVENTION

The invention features methods for diagnosing kidney cancer. In some aspects, the methods comprise determining the concentration of each amino acid in a profile comprising a plurality of amino acids, in a sample of serum obtained from a subject, comparing the determined concentration of each amino acid in the profile with one or more reference concentrations for each amino acid in a reference profile, and determining whether the subject is healthy, is at risk for developing kidney cancer, or has kidney cancer based on the comparison. The methods may further comprise determining the concentration of creatinine in the sample of serum and comparing the determined concentration with one or more reference concentrations for creatinine in a reference profile, and determining whether the subject is healthy, is at risk for developing kidney cancer, or has kidney cancer based on the comparison of both the amino acid and creatinine concentrations. The reference profile may be a reference profile for a healthy subject, a reference profile for a subject at risk for developing kidney cancer, and/or a reference profile for a subject having kidney cancer. The methods are preferably carried out using a processor programmed to compare determined concentrations and reference concentrations, including those for amino acids and/or creatinine. The subject may be any animal, and preferably is a human being.

In some aspects, the reference profile for a subject having kidney cancer comprises one or more of a reference profile for a subject having stage I kidney cancer, a reference profile for a subject having stage II kidney cancer, a reference profile for a subject having stage III kidney cancer, and a reference profile for a subject having stage IV kidney cancer.

The methods may further comprise determining the stage of kidney cancer if the subject has kidney cancer. The methods may further comprise determining the type of kidney cancer. The methods may further comprise determining the subject's prognosis. A prognosis may comprise a substantial likelihood of mortality within about five years, within about three years, within about two years, or within about one year.

The methods may further comprise treating the subject with a treatment regimen capable of improving the prognosis of a kidney cancer patient. The methods may further comprise treating the subject with a treatment regimen capable of inhibiting the advancement of the kidney cancer to a later stage. The methods may further comprise treating the subject with a treatment regimen capable of inhibiting the onset of kidney cancer in a subject at risk for developing kidney cancer. The methods may further comprise treating the subject with a treatment regimen capable of inhibiting recurrence of kidney cancer, for example, in a patient in remission. In any case, the treatment regimen may comprise one or more of surgery, radiation therapy, proton therapy, ablation therapy, hormone therapy, chemotherapy, immunotherapy, stem cell therapy, follow up testing, diet management, vitamin supplementation, nutritional supplementation, exercise, physical therapy, prosthetics, kidney transplantation, reconstruction, psychological counseling, social counseling, education, or regimen compliance management.

Any of the method steps, including optional steps, may be repeated after a period of time. The period of time may be about six months, about one year, about eighteen months, about two years, or about five years. The period between repeats may be shorter than six months or longer than five years. The method steps may be repeated any appropriate number of times.

The invention also features systems for diagnosing kidney cancer. In general, systems comprise a data structure comprising one or more reference profiles comprising one or more reference concentrations for each amino acid in a plurality of amino acids, and optionally comprising one or more reference concentrations for creatinine, and a processor operably connected to the data structure. In preferred aspects, the reference profiles include one or more of a reference profile for a healthy subject, a reference profile for a subject at risk for developing kidney cancer, and a reference profile for a subject having kidney cancer. In preferred aspects, the processor is capable of comparing the concentration of each amino acid in a profile of amino acids determined from a sample of serum obtained from a subject with the reference concentrations. In preferred aspects, the processor is capable of comparing the concentration of creatinine determined from the sample of serum obtained from a subject with the reference creatinine concentrations. In some aspects, a reference profile for a subject having kidney cancer comprises one or more of a reference profile for a subject having stage I kidney cancer, a reference profile for a subject having stage II kidney cancer, a reference profile for a subject having stage III kidney cancer, and/or a reference profile for a subject having stage IV kidney cancer.

The system may further comprise a processor capable of determining the concentration of amino acids in serum obtained from a subject. The system may further comprise an input for accepting the determined concentration of amino acids obtained from the subject. The system may further comprise a processor capable of determining the concentration of creatinine in serum obtained from a subject. The system may further comprise an input for accepting the determined concentration of creatinine obtained from the subject. The system may further comprise an output for providing results of the comparison to a user such as the subject, a technician, or a medical practitioner. The system may further comprise executable code for causing a programmable processor to determine a prognosis of a kidney cancer subject from a comparison of determined amino acid concentrations, and in some aspects, a comparison of determine creatinine concentration, with reference concentrations. The system may further comprise executable code for causing a programmable processor to determine the type of kidney cancer from a comparison of determined amino acid concentrations, and in some aspects, a comparison of determine creatinine concentration, with reference concentrations.

In any of the systems, the processor may be a computer processor. A computer may comprise the processor and the executable code. The system may further comprise a computer network connection such as an Internet connection.

The invention also features computer readable media. In general, computer readable media comprise executable code for causing a programmable processor to compare the concentration of each amino acid in a profile comprising a plurality of amino acids determined from a sample of serum obtained from a subject with one or more reference concentrations for each amino acid in a reference profile. Computer readable media may further comprise executable code for causing a programmable processor to compare the concentration of creatinine determined from a sample of serum obtained from a subject with one or more reference concentrations for creatinine in a reference profile. In preferred aspects, the reference profile comprises one or more of a reference profile for a healthy subject, a reference profile for a subject at risk for developing kidney cancer, and a reference profile for a subject having kidney cancer. In preferred aspects, the reference profile for a subject having kidney cancer comprises one or more of a reference profile for a subject having stage I kidney cancer, a reference profile for a subject having stage II kidney cancer, a reference profile for a subject having stage III kidney cancer, and a reference profile for a subject having stage IV kidney cancer.

The computer readable media may further comprise executable code for causing a programmable processor to determine a prognosis for a kidney cancer patient based on a comparison of determined amino acid concentrations, and in some aspects, a comparison of determined creatinine concentration, with reference concentrations. The computer readable media may further comprise executable code for causing a programmable processor to recommend a treatment regimen for treating a kidney cancer patient. The computer readable media may further comprise a processor.

The executable code of the computer readable media may be capable of causing the programmable processor to recommend a treatment regimen for treating a stage I kidney cancer patient, to recommend a treatment regimen for treating a stage II kidney cancer patient, to recommend a treatment regimen for treating a stage III kidney cancer patient, or to recommend a treatment regimen for treating a stage IV kidney cancer patient.

In any of the methods, systems, or computer readable media, the plurality of amino acids preferably includes alanine, asparagine, arginine, citrulline, cysteine, glutamate, glycine, histidine, methionine, phenylalanine, proline, serine, taurine, threonine, and tyrosine. In some aspects, the plurality of amino acids preferably includes cysteine, histidine, leucine, lysine, ornithine, proline, tyrosine, and valine.

In any of the methods, systems, or computer readable media, the kidney cancer may be renal cell carcinoma or transitional cell carcinoma. Preferred examples of renal cell carcinoma include clear cell renal cell carcinoma, papillary type I renal cell carcinoma, papillary type II renal cell carcinoma, chromophobe renal cell carcinoma, collecting duct renal cell carcinoma, oncocyte renal cell carcinoma, and unclassified renal cell carcinoma. Preferred examples of transitional cell carcinoma include Wilms' tumor and renal sarcoma.

### BRIEF DESCRIPTION OF THE DRAWINGS

FIG. 1 shows a trace file of human plasma from a BioChrom® 30 amino acid analyzer. The x-axis shows the elution time in minutes after injection. The y-axis shows relative absorbance at 570 nm.

FIG. 2 shows a correlation of amino acids in a data set.

FIG. 3 shows receiver operator curves (ROC) for a logistic regression model. FIG. 3A shows a ROC for a logistic regression model presented in Table 3. Samples include all patients (n=190) and all controls (n=104). FIG. 3B shows a ROC for only early stage patients (n=112) and all controls (n=104).

FIG. 4 shows Patient Logistic Regression Model Scores stratified by tumor grade and type. FIG. 4A shows a Logistic Regression Model Score stratified by tumor grade; the mean score for each grade is shown. Error bars show 95% confidence interval of mean. Stage 0 are control samples. FIG. 4B shows a Logistic regression model score stratified by tumor type.

FIG. 5 shows survival curves stratified by logistic regression model score. FIG. 5A shows Kaplan Meier's curves for all RCC patients (n=190) stratified by logistic regression score either being above or below the median (0.79, P<0.0045). FIG. 5B shows Kaplan Meier's curves for only stage 4 patients (n=40, P=0.049) stratified by logistic regression score either being above or below 0.72.

FIG. 6 shows a receiver operator curve of the logistic regression model shown in Table 4 combined with determined serum creatinine levels (Mod+Cre). The addition of creatinine levels increased the area under the ROC from 0.8080 (FIG. 3B) to 0.8470.

FIG. 7 shows the overall survival based on the Mod+Cre score. The top line (Group j) shows overall survival of patients with a score above the patient mean, and the bottom line (Group 1) shows survival of patients with a score below the mean.

FIG. 8 shows a non-limiting example of a system for diagnosing kidney cancer.

### DETAILED DESCRIPTION OF THE INVENTION

Various terms relating to aspects of the invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the,” include plural referents unless expressly stated otherwise.

The terms measure or determine are used interchangeably, and refer to any suitable qualitative or quantitative determinations.

The terms subject or patient are used interchangeably. A subject may be any animal, including mammals such as companion animals, laboratory animals, and non-human primates. Human beings are preferred.

Statistically significant changes in the levels of 15 different amino acids were observed in the serum of renal cell carcinoma patients as compared with age- and sex-matched healthy controls. In accordance with the invention, a model was developed using these amino acids that may be used to differentiate between kidney cancer patients and healthy subjects and to differentiate between early stage and later stage kidney cancer, as well as to predict survival of kidney cancer patients. It was observed that the predictive power of the model, including the capacity to predict patient survival, could be enhanced by measuring serum creatinine concentration and including creatinine with the amino acids. The model thus may be used as a diagnostic and prognostic tool, including for identifying patients with recurrent cancer. Accordingly, the invention features computer readable media, systems, and methods for diagnosing kidney cancer, for characterizing the stage of kidney cancer, for providing a prognosis of kidney cancer patients, and for establishing and refining a kidney cancer treatment regimen.

In one aspect, the invention features methods for diagnosing kidney cancer. In general, the methods comprise determining the concentration of each amino acid in a profile comprising a plurality of amino acids, the concentration of each amino acid in the profile being determined from a sample of blood or serum obtained from a subject, comparing the determined concentration of each amino acid in the profile with one or more reference concentrations for each amino acid in a reference profile, and based on this comparison, determining whether the subject is healthy, is at risk for developing kidney cancer, or has kidney cancer. The methods may further comprise determining the concentration of creatinine in the sample of blood or serum obtained from the subject, and comparing the determined concentration of creatinine with one or more reference concentrations for creatinine, and based on the combined comparison of amino acid and creatinine concentrations, determining whether the subject is healthy, is at risk for developing kidney cancer, or has kidney cancer. Each comparing step may be carried out using a processor programmed to compare determined concentrations with reference concentrations. In preferred aspects, the amino acids in the determined profile and the amino acids in the reference profiles are the same.

The reference profiles may comprise one or more reference profiles for a healthy subject, reference profiles for a subject at risk for developing kidney cancer, and reference profiles for a subject having kidney cancer. The U.S. National Cancer Institute classifies cancer according to four basic stages: Stage I, Stage II, Stage III, and Stage IV, based on the TNM scoring system (Primary Tumor, Regional Lymph Nodes, and Distant Metastasis). Thus, the reference profiles may comprise one or more reference profiles for a subject having stage I kidney cancer, reference profiles for a subject having stage II kidney cancer, reference profiles for a subject having stage III kidney cancer, and reference profiles for a subject having stage IV kidney cancer.

Reference profiles may comprise reference concentrations of amino acids obtained or derived from population studies, for example, population reference profiles. Reference profiles may comprise reference concentrations of creatinine obtained or derived from population studies. It is contemplated that over time, additional studies will generate new and additional information about the serum amino acid and/or creatinine profiles and amino acid and creatinine concentrations for healthy subjects, kidney cancer subjects and the stages thereof, subjects having recurrent kidney cancer, and subjects at risk for developing kidney cancer and at risk for developing recurrent kidney cancer. The additional information may increase the accuracy, reliability, and confidence of the reference profiles, and accordingly increase the accuracy, reliability, and confidence of the determinations and recommendations realized by carrying out the methods. Thus, newly generated or revised reference concentrations and reference profiles may be used in accordance with the methods, systems, and computer readable media described and exemplified herein.

Reference profiles may comprise reference concentrations of amino acids obtained previously from the subject. Reference profiles may comprise reference concentrations of creatinine obtained previously from the subject. For example, a blood or serum amino acid concentration profile, which may include serum creatinine concentration, generated from the subject may be compared against a blood or serum amino acid concentration profile, which may include serum creatinine concentration, previously generated from the subject. The profile may comprise a plurality of amino acids. The previously generated profile may comprise a healthy profile, an at-risk profile, a positive kidney cancer profile, or a profile of a particular stage of kidney cancer. Thus, the amino acid and creatinine concentrations in the later-generated reference profile may be compared against the amino acid and creatinine concentrations in the earlier-generated reference profile. The comparison may be used to monitor the subject over time, for example, to determine the level of response to a particular treatment regimen, or to determine any change in the subject's condition such as a change from a healthy state to an at-risk or precancerous state or cancerous state, or an at-risk or precancerous state to a cancerous state. The comparison may also be used to determine if cancer has recurred in the subject.

In preferred aspects, the plurality of amino acids comprises amino acids whose concentrations are altered in subjects at risk for kidney cancer relative to healthy subjects, or that are altered in subjects who have kidney cancer relative to subjects at risk for kidney cancer and/or healthy subjects. Additionally, the plurality of amino acids may comprise amino acids whose concentrations are altered in subjects in a late stage of kidney cancer relative to subjects in an early stage of kidney cancer or relative to healthy subjects, or subjects in an early stage of kidney cancer relative to healthy subjects. Additionally, the reference amino acid concentrations may include those whose concentrations indicate that the cancer has recurred. Non-limiting examples of amino acids that may be included within the plurality include alanine, asparagine, arginine, citrulline, cysteine, glutamate, glycine, histidine, leucine, lysine, methionine, ornithine, phenylalanine, proline, serine, taurine, threonine, tyrosine, and valine. A plurality may include any number or combination of amino acids. A preferred plurality includes alanine, asparagine, arginine, citrulline, cysteine, glutamate, glycine, histidine, methionine, phenylalanine, proline, serine, taurine, threonine, and tyrosine. A preferred plurality includes cysteine, histidine, leucine, lysine, ornithine, proline, tyrosine, and valine.

In preferred aspects, the reference creatinine concentrations include those that are altered in subjects at risk for kidney cancer relative to healthy subjects, or that are altered in subjects who have kidney cancer relative to subjects at risk for kidney cancer and/or healthy subjects. Additionally, the reference creatinine concentrations may include those whose concentrations are altered in subjects in a late stage of kidney cancer relative to subjects in an early stage of kidney cancer or relative to healthy subjects, or subjects in an early stage of kidney cancer relative to healthy subjects. Additionally, the reference creatinine concentrations may include those whose concentrations indicate that the cancer has recurred.

Optionally, the methods may comprise determining the stage of kidney cancer. Optionally, the methods may comprise determining the particular kidney cancer. In any case, the kidney cancer may be renal cell carcinoma or transitional cell carcinoma. Non-limiting examples of renal cell carcinoma include clear cell renal cell carcinoma, papillary type I renal cell carcinoma, papillary type II renal cell carcinoma, chromophobe renal cell carcinoma, collecting duct renal cell carcinoma, oncocyte renal cell carcinoma, or unclassified renal cell carcinoma. Non-limiting examples of transitional cell carcinoma include Wilms' tumor or renal sarcoma.

Serum amino acid concentration profiles, which may include serum creatinine concentration, may be used to determine a likelihood of survival. Thus, the methods may optionally comprise determining the subject's prognosis based on the comparison of the measured profile of amino acid concentrations in the subject's blood or serum with the one or more reference profiles. The methods may optionally comprise determining the subject's prognosis based on the comparison of the measured profile of amino acid concentrations in the subject's blood or serum with the one or more reference profiles for amino acid concentrations and based on the comparison of the measured creatinine concentration in the subject's blood or serum with reference concentrations for creatinine.

A prognosis may relate to, or be measured according to any time frame. For example, the prognosis may comprise a substantial likelihood of mortality within about five years. The prognosis may comprise a substantial likelihood of mortality within about three years. The prognosis may comprise a substantial likelihood of mortality within about two years. The prognosis may comprise a substantial likelihood of mortality within about one year. In some aspects, the prognosis may comprise an about two to about five year range of time. The prognosis may comprise an about three to about five year range of time. The prognosis may comprise an about three to about ten year range of time. The prognosis may comprise an about five to about ten year range of time. Time frames may be shorter than one year or may be longer than five years. Time frames may vary according to clinical standards, or according to the needs or requests from the patient or practitioner.

Optionally, the methods may comprise treating the subject with a regimen capable of improving the prognosis of a kidney cancer patient. In the case of a subject determined to be at risk for developing a kidney cancer, the methods may comprise treating the subject with a regimen capable of preventing, inhibiting, or otherwise slowing the development of kidney cancer. For subjects determined to have an early stage kidney cancer, the methods may comprise treating the subject with a regimen capable of preventing, inhibiting, or otherwise slowing the advancement of the kidney cancer to a later stage. For subjects that may be at risk for recurrence, the methods may comprise treating the subject with a regimen capable of preventing, inhibiting, or otherwise slowing the recurrence of kidney cancer in a patient in remission.

The regimen may be tailored to the specific characteristics of the subject, for example, the age, sex, or weight of the subject, the type or stage of the cancer, and the overall health of the subject. The regimen may comprise one or more of surgery, radiation therapy, proton therapy, ablation therapy, hormone therapy, chemotherapy, immunotherapy, stem cell therapy, follow up testing, diet management, vitamin supplementation, nutritional supplementation, exercise, physical therapy, kidney transplantation, reconstruction, psychological counseling, social counseling, education, and regimen compliance management. Suitable treatments for Kidney cancer include administering to the subject an effective amount of interleukin-2, alpha-interferon, bevacizumab, sutent, sorafenib, pazopanib, everolimus, and/or temsirolimus.

The steps of the methods, including any optional steps, may be repeated after a period of time, for example, as a way to monitor a subject's health and prognosis. Thus for example, in some aspects, the methods optionally further comprise repeating the determining and comparing steps after a period of time. Repeating the methods may be used, for example, to determine if a subject has advanced from a healthy state to a precancerous or cancerous state. Repeating the methods may be used, for example, to determine if a subject has recurrent cancer. Repeating the methods may be used, for example, to determine if the patient's prognosis has improved based on a particular treatment regimen, or to determine if adjustments to the treatment regimen should be made to achieve improvement or to attain further improvement in the patient's prognosis. The methods may be repeated at least one time, two times, three times, four times, or five or more times. The methods may be repeated as often as the patient desires, or is willing or able to participate.

The period of time between repeats may vary, and may be regular or irregular. In some aspects, the methods are repeated in three month intervals. In some aspects, the methods are repeated in six month intervals. In some aspects, the methods are repeated in one year intervals. In some aspects, the methods are repeated in two year intervals. In some aspects, the methods are repeated in five year intervals. In some aspects, the methods are repeated only once, which may be about three months, six months, twelve months, eighteen months, two years, three years, four years, five years, or more from the initial assessment.

Optionally, the methods may comprise the step of obtaining a sample of blood or serum from a subject. In aspects where blood is obtained, serum may be isolated from the blood. Blood or serum may be obtained from a subject according to any means suitable in the art.

The invention also features systems 10 for diagnosing kidney cancer. See, e.g., FIG. 8. In general, such systems 10 comprise a data structure 20 that comprises a plurality of reference profiles comprising one or more reference concentrations of each amino acid in a plurality of amino acids, and a programmable processor 22 such as a computer operably connected to the data structure 20. The data structure 20 may further comprise one or more reference concentrations for creatinine. Such reference profiles may include reference profiles for a healthy subject, reference profiles for a subject at risk for developing kidney cancer, reference profiles for a subject having kidney cancer, reference profiles for a subject having stage I kidney cancer, reference profiles for a subject having for stage II kidney cancer, reference profiles for a subject having stage III kidney cancer, and reference profiles for a subject having stage IV kidney cancer. Preferably, the processor 20 is capable of comparing the concentration of each amino acid in the profile of amino acids determined from a sample of blood or serum obtained from a subject with the reference concentrations of amino acids in the one or more reference profiles. The processor 20 may also be capable of comparing the concentration of creatinine determined from the sample of blood or serum obtained from the subject with the reference concentrations of creatinine. The processor 20 preferably is a computer processor. The systems 10 may comprise a graphical user interface.

In preferred aspects, the plurality of amino acids comprises amino acids whose concentrations are altered in subjects at risk for kidney cancer relative to healthy subjects, or that are altered in subjects who have kidney cancer relative to subjects at risk for kidney cancer and/or healthy subjects. Additionally, the plurality of amino acids may comprise amino acids whose concentrations are altered in subjects in a late stage of kidney cancer relative to subjects in an early stage of kidney cancer or relative to healthy subjects, or subjects in an early stage of kidney cancer relative to healthy subjects. Non-limiting examples of amino acids that may be included within the plurality include alanine, asparagine, arginine, citrulline, cysteine, glutamate, glycine, histidine, leucine, lysine, methionine, ornithine, phenylalanine, proline, serine, taurine, threonine, tyrosine, and valine. A plurality may include any number or combination of amino acids. A preferred plurality includes alanine, asparagine, arginine, citrulline, cysteine, glutamate, glycine, histidine, methionine, phenylalanine, proline, serine, taurine, threonine, and tyrosine. A preferred plurality includes cysteine, histidine, leucine, lysine, ornithine, proline, tyrosine, and valine.

In some aspects, the system 10 optionally comprises a processor 20 capable of determining the concentration of amino acids, for example, a profile of amino acids, in blood or serum obtained from a subject. The processor 20 may be capable of determining the concentration of creatinine in the blood or serum. Such a processor 20 may be the same processor 20 as the processor 20 capable of comparing determined amino acid concentrations with reference concentrations, or may be a separate processor. The processor 20 is preferably a computer processor.

Optionally, the systems 10 may comprise an input 24 for accepting data, such as determined amino acid and creatinine concentrations, entered into the system. The systems 10 may comprise an output 26 for providing information to a user. Such information may, for example, a diagnosis and/or a prognosis. The user may be a patient or a medical practitioner. The systems 10 may be used to carry out any method described or exemplified herein.

Optionally, the system 10 may comprise executable code for causing a programmable processor 20 to determine a diagnosis of the subject, for example whether the subject is healthy, is at risk for kidney cancer, has kidney cancer, and the type or stage of kidney cancer, which determination may be based on the comparison of measured amino acid concentrations with reference amino acid concentrations, as well as a comparison of measured creatinine concentration with reference creatinine concentrations. Optionally, the system 10 may comprise executable code for causing a programmable processor 20 to determine a prognosis of the subject. The executable code for determining a diagnosis and the executable code for determining a prognosis may comprise the same executable code, or may comprise separate executable code.

In any of the systems 10, a computer may comprise the programmable processor or processors 20 used for determining information, comparing information and determining results. The computer may comprise the executable code for determining a diagnosis of the subject, and/or may comprise the executable code for determining a prognosis of the subject. The systems 10 may comprise a computer network connection 28, including an Internet connection 28.

The invention also features computer-readable media. The media may be used with the systems and/or methods. In general, computer readable media comprise executable code for causing a programmable processor to compare the concentration of each amino acid in a profile comprising a plurality of amino acids determined from a sample of blood or serum obtained from a subject with one or more reference concentrations for each amino acid in a reference profile. The computer readable media may further comprise executable code for causing a programmable processor to compare the concentration of creatinine determined from the sample of blood or serum obtained from the subject with one or more reference concentrations for creatinine. The computer readable media may comprise a processor, which may be a computer processor.

In preferred aspects, the reference profile comprises one or more of a reference profile for a healthy subject, a reference profile for a subject at risk for developing kidney cancer, and a reference profile for a subject having kidney cancer. The reference profile for a subject having kidney cancer preferably comprises one or more reference profiles for a subject having stage I kidney cancer, reference profiles for a subject having stage II kidney cancer, reference profiles for a subject having stage III kidney cancer, and reference profiles for a subject having stage IV kidney cancer.

In preferred aspects, the plurality of amino acids comprises amino acids whose concentrations are altered in subjects at risk for kidney cancer relative to healthy subjects, or that are altered in subjects who have kidney cancer relative to subjects at risk for kidney cancer and/or healthy subjects. Additionally, the plurality of amino acids may comprise amino acids whose concentrations are altered in subjects in a late stage of kidney cancer relative to subjects in an early stage of kidney cancer or relative to healthy subjects, or subjects in an early stage of kidney cancer relative to healthy subjects. Non-limiting examples of amino acids that may be included within the plurality include alanine, asparagine, arginine, citrulline, cysteine, glutamate, glycine, histidine, leucine, lysine, methionine, ornithine, phenylalanine, proline, serine, taurine, threonine, tyrosine, and valine. A plurality may include any number or combination of amino acids. A preferred plurality includes alanine, asparagine, arginine, citrulline, cysteine, glutamate, glycine, histidine, methionine, phenylalanine, proline, serine, taurine, threonine, and tyrosine. A preferred plurality includes cysteine, histidine, leucine, lysine, ornithine, proline, tyrosine, and valine.

Optionally, the computer readable media may comprise executable code for causing a programmable processor to determine a prognosis for a kidney cancer patient based on a comparison of amino acid concentrations determined from samples of blood or serum obtained from a subject and reference concentrations comprised in reference profiles. Optionally, the computer readable media may comprise executable code for causing a programmable processor to determine a prognosis for a kidney cancer patient based on a comparison of amino acid concentrations determined from samples of blood or serum obtained from a subject and creatinine concentration determined from the samples of blood or serum with reference concentrations of amino acids and creatinine. The reference concentrations of amino acids may be comprised in reference profiles. Optionally, the computer readable media may comprise executable code for causing a programmable processor to determine the type and/or stage of kidney cancer. Optionally, the computer readable media may comprise executable code for causing a programmable processor to recommend a treatment regimen for treating a kidney cancer patient. The executable code may be capable of causing a programmable processor to recommend a treatment regimen for treating a stage I kidney cancer patient, a stage II kidney cancer patient, a stage III kidney cancer patient, and/or a stage IV kidney cancer patient. The treatment regimen may be any regimen known in the art, including those described herein. The kidney cancer may be renal cell carcinoma or transitional cell carcinoma.

The following examples are provided to describe the invention in greater detail. They are intended to illustrate, not to limit, the invention.

### Example 1

### Amino Acid Profiling Methods

Patients and Samples. Blood serum for analysis was obtained from Renal Cell Carcinoma (RCC) patients and control samples were obtained from an in-house repository. After receiving each RCC patient's consent, blood was collected, and serum was isolated and stored at −70° C. All samples were collected between 2004 and 2010. Control serums stored at the repository came from a variety of sources including in-house employees, individuals undergoing routine cancer screening, and spouses of cancer patients. Controls were selected by matching each of the first 104 cases by age and sex.

Amino Acid analysis. Five microliters of 12% dithiothreitol (DTT) were added to fifty microliters of plasma, and samples were incubated at room temperature for 5 minutes to reduce the samples. Next, 55 microliters of 10% sulfosalicylic acid were added to the plasma-DTT mix, and the samples were incubated for one hour at 4° C. Samples were then centrifuged at 12,000×g for ten minutes and the supernatant was collected and loaded into auto-loading tubes. Auto-loading tubes were fed into a BioChrom® 30 (BioChrom Ltd. Corp., Cambridge, UK) amino acid analyzer and peaks were identified and quantitated using EZ Lite software. Quantitation of the different amine-containing compounds was determined by comparing peak area to a known standard. Inter-day assay repeatability was established by processing 27 different samples on two different days and calculating the co-efficient of variation for each of the 26 amino acids quantitated in each of the 27 pairs of samples tested. The average coefficient of variation (CV) for all of the amino acids was 6.7% (range 3.5-14.2%).

Data Analysis. Data analysis was performed using Statistica 9.1 software (Statsoft, Tulsa Okla.). If necessary, data was log-transformed to ensure normal distribution. For univariate analysis, two-sided t-tests were used. For multiple group analysis ANOVA was used.

To determine if amino acid analysis can effectively identify cases from controls, backward logistic regression was performed using all 26 amino acids as variables. All variables with P<0.05 were included in the final model.

### Example 2

### Amino Acid Profiling Results

Patient and Control Characteristics. Serum was obtained from 190 RCC patients at the investigator's clinical facilities between the years of 2004 and 2010 before undergoing a nephrectomy. The characteristics of the patients are shown on Table 1. The median age of the patients was 58 years old, with the majority of the patients being male and white. Control samples were obtained from an in-house biosample repository by individually matching for sex, race and age for the first 104 patient samples obtained. No significant differences were found in the distribution of age, sex, race or body mass index (BMI) between the control and patient group as a whole.

TABLE 1
Characteristics of RCC cases and controls

| | | Case (n = 190) | | Control (n = 104) | | P value |
|---|---|---|---|---|---|---|
| Age | Median | 58 | | 57 | | 0.49 |
| | Range | (25-87) | | (36-81) | | |
| Sex | Male | 137 | (72%) | 71 | (69%) | 0.93 |
| | Female | 53 | (28%) | 32 | (31%) | |
| BMI | | 29.8 | (n = 61) | 27.6 | (n = 97) | 0.09 |
| Race | White | 156 | (82%) | 93 | (89%) | 0.97 |
| | Black | 17 | (08%) | 8 | (07%) | |
| | Asian | 1 | (0.5%) | 1 | (0.9%) | |
| | Unknown | 16 | (8.4%) | 2 | (1.9%) | |
| Stage | I | 100 | (53%) | | | |
| | II | 23 | (12%) | | | |
| | III | 27 | (14%) | | | |
| | IV | 40 | (21%) | | | |
| Type | CRCC | 120 | (63%) | | | |
| | PRC | 29 | (15%) | | | |
| | Other | 41 | (22%) | | | |

| | | |
|---|---|---|
| Total | 190 | 104 |

Abbreviations:
BMI, Body Mass Index;
CRCC, clear renal cell carcinoma;
PRC, papillary renal carcinoma;
Other includes adenocarcinoma with mixed subtype (15), chromophobe (13), cyst associated (4), sarcomatoid (2), carcinoma (2), small cell (2), granular cell (1).

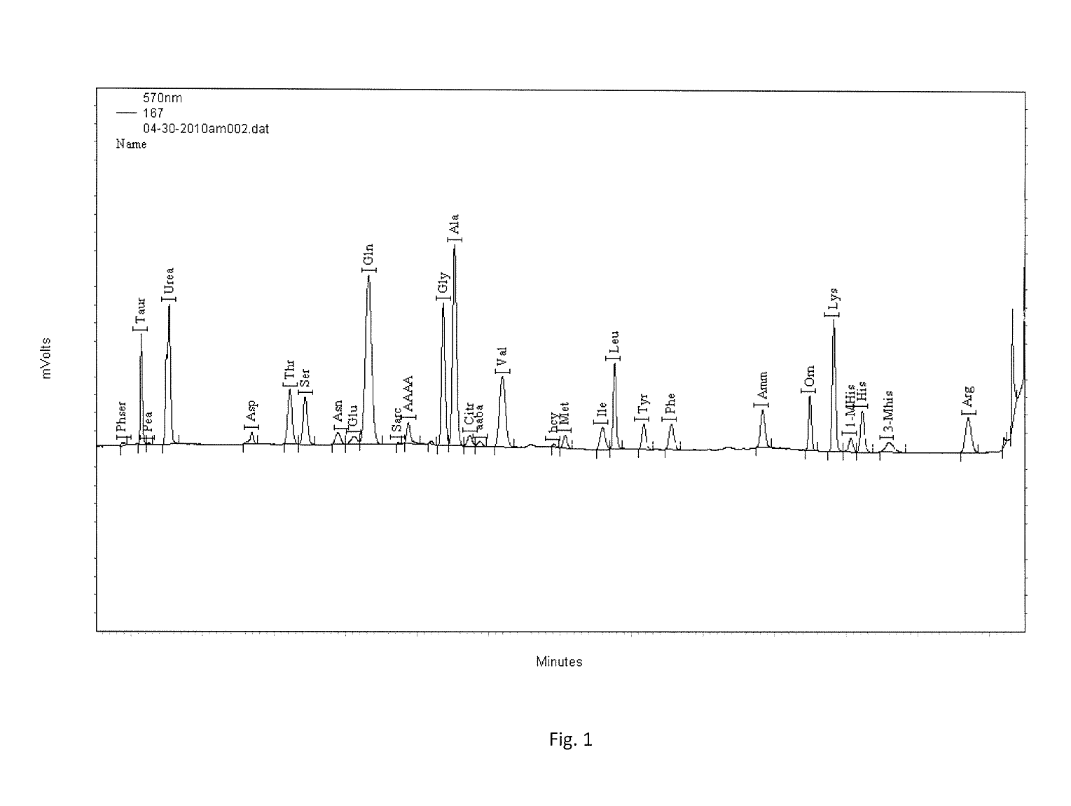

Amino Acid analysis. Each patient and control serum sample was analyzed for amino acid content using an amino acid analyzer. Twenty-six compounds were quantitated for each sample including taurine, aspartate, threonine, serine, asparagines, glutamate, glutamine, glycine, alanine, citrulline, alpha-amino butyrate, valine, homocysteine, methionine, isoleucine, leucine, tyrosine, phenylalanine, ornithine, lysine, 1-methylhistidine, histidine, 3-methylhistidine, arginine, cysteine, and proline (FIG. 1).

Comparison of patients and controls revealed that 15 of the 26 amino acids examined showed statistically significant differences in the means between cases and controls (Table 2). Twelve (taurine, threonine, serine, asparagines, glutamate, glycine, alanine, citrulline, methionine, tyrosine, phenylalanine, histidine, and proline) were significantly decreased in RCC patients, and two (arginine and cysteine) were elevated. The largest percent differences between the means were observed for histidine and ornithine. Since most of the significantly changed amino acids appeared to be lower in the RCC patients relative to controls, the hypothesis that this might be due to decreased kidney function in the RCC patients was tested. However, pre-operative glomular filtration rates (GFR) in patients were not significantly correlated with amino acid levels, with the exception of citrulline, homocysteine, and 1-methyl histidine.

TABLE 2
Amino Acid Mean and t-Test for Cases vs. Control.

| | Case | | Control | | p | |
|---|---|---|---|---|---|---|
| | (n = 190) | | (n = 104) | | T-test | |
| Amino Acid | Mean | Std | Mean | Std | 2-sided | p^{Adjusted} |
| Taurine | 159.4 | 52.4 | 174.3 | 58.2 | 0.0265 | .681 |
| Aspartate | 32.4 | 14.3 | 35.9 | 16.8 | 0.0672 | .685 |
| Threonine | 134.7 | 40.1 | 153.6 | 40.4 | 0.0001 | .013 |
| Serine | 132.1 | 33.3 | 142.9 | 41.0 | 0.0156 | .680 |
| Asparagine | 68.3 | 19.5 | 78.1 | 25.8 | 0.0003 | .229 |
| Glutamate | 98.9 | 56.9 | 129.7 | 102.4 | 0.0010 | .732 |
| Glutamine | 854.7 | 182.1 | 867.0 | 213.3 | 0.6029 | .178 |
| Glycine | 287.9 | 80.5 | 321.1 | 110.9 | 0.0036 | .256 |
| Alanine | 451.6 | 122.4 | 527.5 | 163.3 | 0.0000 | .003 |
| Citrulline | 34.7 | 12.2 | 38.4 | 9.7 | 0.0082 | .061 |
| alpha-amino | 21.3 | 9.3 | 21.0 | 10.7 | 0.7951 | .017 |
| butyric acid | | | | | | |
| Valine | 254.1 | 58.8 | 268.0 | 66.6 | 0.0653 | .238 |
| tHomocysteine | 14.5 | 6.6 | 15.4 | 9.4 | 0.3774 | .052 |
| Methionine | 23.7 | 6.5 | 25.7 | 8.0 | 0.0168 | .733 |
| Isoleucine | 67.8 | 19.8 | 69.3 | 22.8 | 0.5393 | .006 |
| Leucine | 156.5 | 39.0 | 161.6 | 47.0 | 0.3205 | .001 |
| Tyrosine | 66.9 | 18.2 | 74.5 | 19.8 | 0.0010 | .204 |
| Phenylalanine | 79.0 | 19.5 | 86.5 | 44.8 | 0.0479 | .129 |
| Ornithine | 97.8 | 32.4 | 126.3 | 55.2 | 0.0000 | .000001 |
| Lysine | 206.1 | 50.7 | 217.4 | 53.7 | 0.0766 | .081 |
| 1-methyl- | 19.1 | 13.8 | 18.3 | 10.5 | 0.5782 | .358 |
| histidine | | | | | | |
| Histidine | 77.4 | 19.7 | 90.0 | 22.2 | 0.0000 | .00002 |
| 3-methyl- | 22.9 | 6.1 | 24.0 | 5.8 | 0.1100 | .680 |
| histidine | | | | | | |
| Arginine | 98.7 | 31.1 | 84.0 | 33.8 | 0.0002 | .000018 |
| tCysteine | 401.8 | 98.2 | 374.5 | 87.6 | 0.0185 | .000001 |
| Proline | 214.3 | 83.2 | 230.9 | 63.8 | 0.0774 | .373 |
| Factor 1 | 0.130 | 0.934 | −0.237 | 1.075 | 0.0025 | NA |
| Factor 2 | −0.070 | 0.863 | 0.127 | 1.205 | 0.1061 | NA |
| Factor 3 | 0.027 | 1.018 | −0.050 | 0.968 | 0.530 | NA |

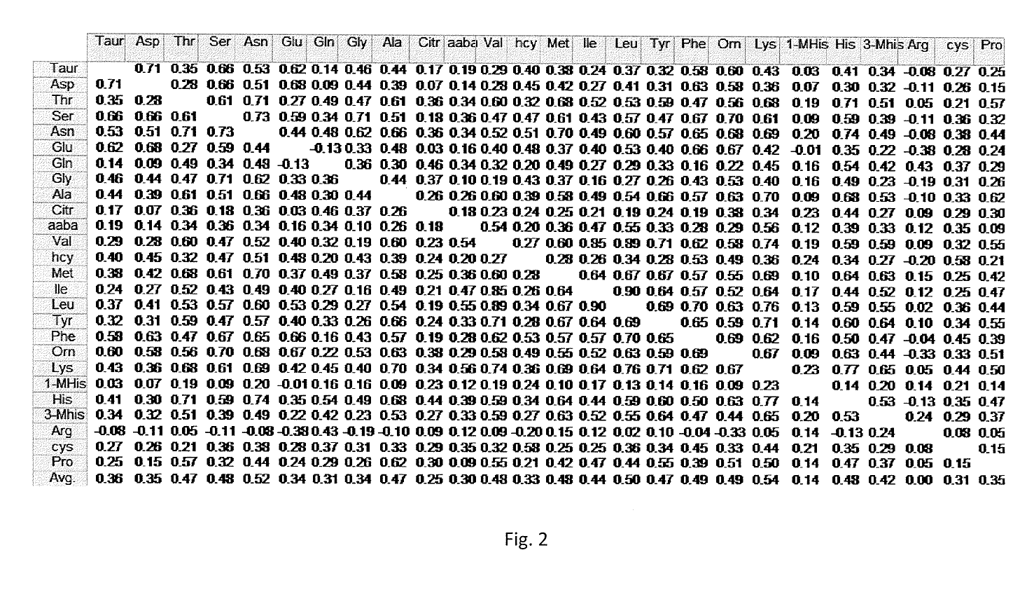

Whether the levels of different amino acids were correlated with each other in the entire dataset was also examined (FIG. 2). With the exception of arginine, there was a statistically significant positive correlation between most of the different amino acid pairs, with the strength of the correlation varying depending on the pairs examined. The strongest correlations were between leucine, isoleucine, and valine (R=0.85-0.89), while the mean correlation co-efficient (R) between different amino acids excluding arginine was 0.39. Factor analysis indicated that a single primary factor could explain 45% of the variance in amino acid levels, and the first three factors together could explain 62.6% of the variance. However, only the primary factor was shown to be significantly different between cases and controls (Table 2).

Because of the significant correlation between different amino acids and the strength of the primary factor, it was possible that some of the significant differences observed in univariate t-tests might be due to this underlying general correlation. Therefore, to control for this, the significance value in which each amino acid was adjusted for this factor was also determined (Table 2, P^{adjusted}). When adjusted in this way, nine amino acids including threonine, alanine, alpha-aminobutyrate, isoleucine, leucine, ornithine, histidine, arginine and cysteine still showed significant differences between cases and controls. Thus, these amino acids are significantly different in cases and controls independent of any general amino acid effect.

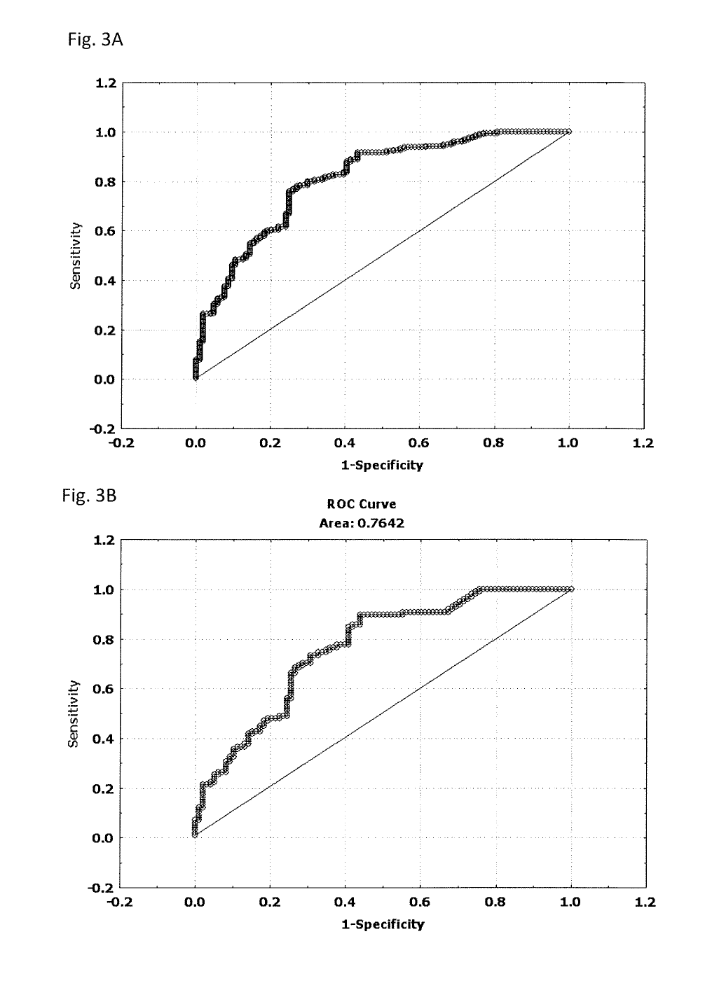

Logistic Regression Model. A logistic regression model that could distinguish cases from controls was created. To create the model a backward-stepwise procedure was performed to identify which of the twenty-six amino acids had significant predictive value (P<0.05) with regard to a sample being either a case or control. The final model contained eight different amino acids (cysteine, ornithine, histidine, leucine, tyrosine. proline, valine, and lysine), and the receiver-operator curve (ROC) for this model gave an AUC 0.81 (Table 3, FIG. 3).

TABLE 3
Logistic Regression Model

| Predictor | Beta | SE Beta | Wald's χ² | p | e^{Beta} |
|---|---|---|---|---|---|
| Intercept | 0.5184 | 0.7995 | 0.4205 | 0.516704 | NA |
| Cys | 0.0061 | 0.0142 | 21.256 | 0.000004 | 1.0061 |
| Orn | −0.0525 | 0.0115 | 20.908 | 0.000005 | 0.9489 |
| His | −0.1160 | 0.0275 | 17.739 | 0.000025 | 0.8905 |
| Leu | 0.0426 | 0.0117 | 13.352 | 0.000256 | 1.0435 |
| Tyr | −0.0355 | 0.0142 | 6.2822 | 0.012196 | 0.9651 |
| Val | −0.0159 | 0.0069 | 5.3491 | 0.020723 | 0.9842 |
| Pro | 0.0069 | 0.0031 | 5.1346 | 0.023454 | 1.007 |
| Lys | 0.0252 | 0.0125 | 4.1001 | 0.042881 | 1.0255 |

Hosmar & Lemeshow test: p=0.6687

Because the number of potential predictor variables in the model (26) was relatively large compared to the total number of samples (290), there was a concern about the model over-fitting the data. To address this possibility, a 10-fold cross validation was performed on the sample set. This procedure involves using 90% of the data set as the analysis group (used to build the model) and 10% as the validation group. This procedure was then performed 10-different times using a unique validation group in each iteration. Performing this procedure using the eight amino acids identified above to make the model showed using ROC analysis that the mean AUC for the analysis group vs. the validation group was not significantly different (0.81 vs. 0.79, p=0.17, Table 4). This result indicates that the model is not over-fitting the data to a significant degree.

TABLE 4
10-fold cross validation testing.

| Run # | Analysis AUC | Validation AUC |
|---|---|---|
| 1 | 0.801 | 0.8181 |
| 2 | 0.7994 | 0.8722 |
| 3 | 0.8133 | 0.7792 |
| 4 | 0.8139 | 0.7833 |
| 5 | 0.8191 | 0.7355 |
| 6 | 0.7987 | 0.8472 |
| 7 | 0.8114 | 0.7613 |
| 8 | 0.8089 | 0.7828 |
| 9 | 0.8231 | 0.6985 |
| 10 | 0.8076 | 0.8051 |
| Avg. | 0.80964 | 0.78832 |
| t-test | 0.17808133 | |

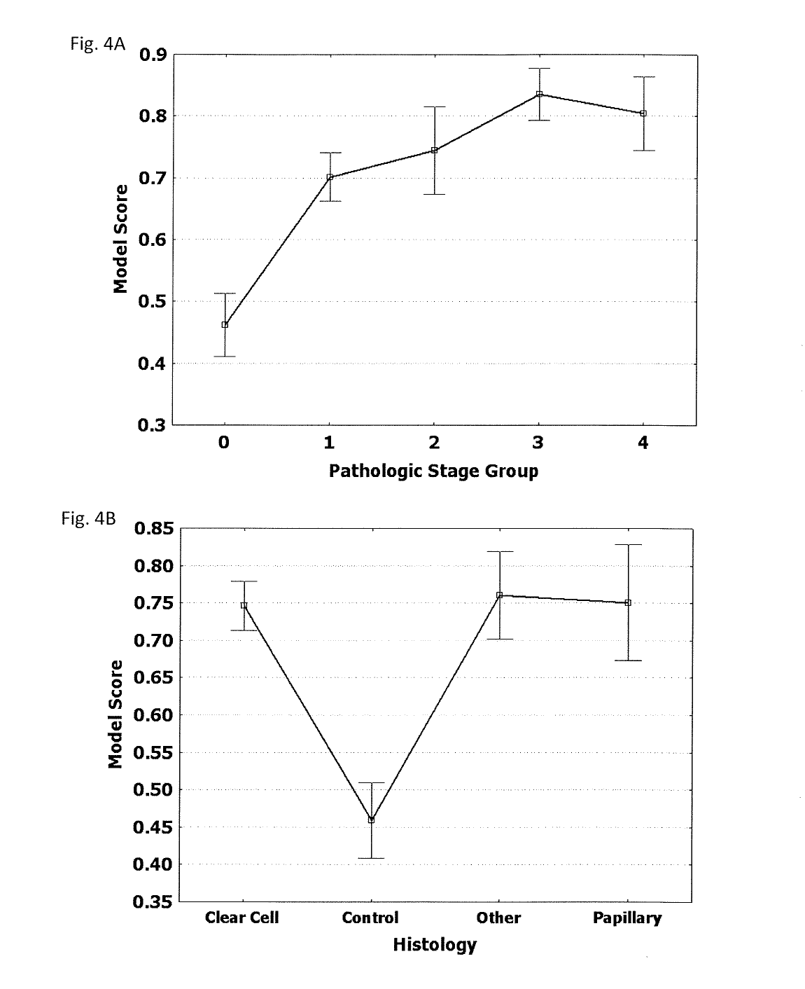

Model Performance on tumor grade and type. Performance of the model relative to pathologic tumor stage was next evaluated. First, the mean predicted value for the samples relative to their tumor grade (FIG. 4a) was examined. As shown in the figure, early stage tumors (stage I and stage II) have slightly lower model scores than late stage tumors (stage III and stage IV), but are still significantly elevated relative to the control samples. ROC analysis on only stage I and stage 2 samples gives an AUC of 0.76, only slightly lower than the total data set (FIG. 3b). Performed of the model on different histological subtypes of kidney cancer was also analyzed (FIG. 4b). The mean value was not significantly different between clear cell, papillary, and a mixture of other types of kidney tumors.

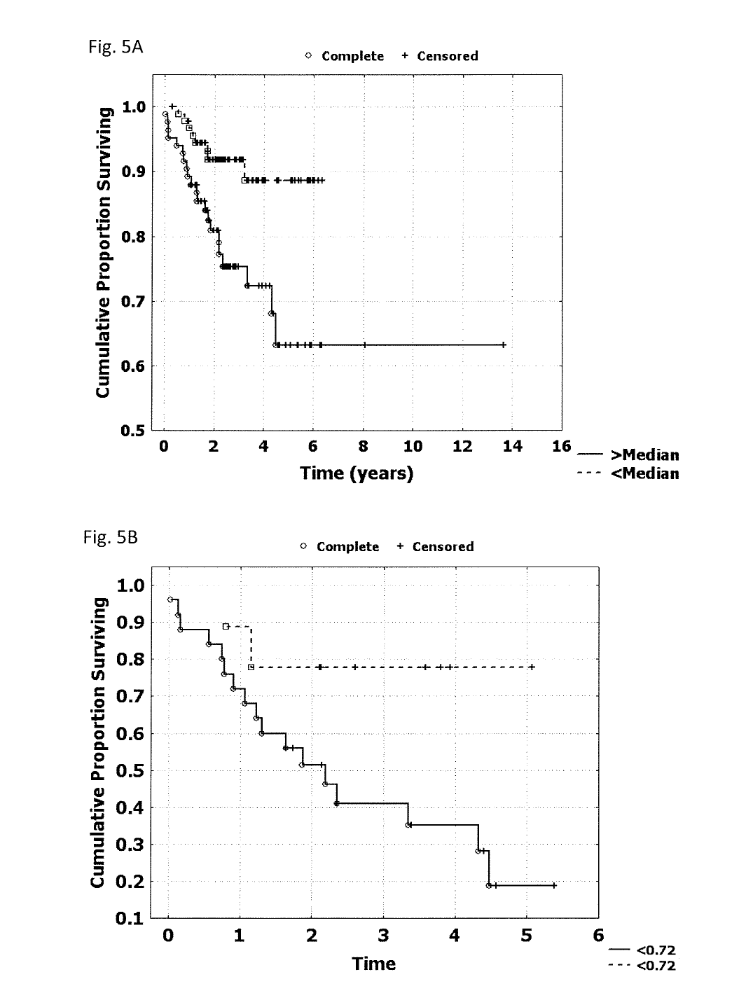

Serum amino acid profiles and survival. The logistic regression score on patient samples was next related to overall survival. For this analysis patients were divided into two groups, those with logistic regression scores above and below the median (0.79). It was found that patients with lower logistic regression scores had significantly increased overall survival compared to those with higher scores (p=0.0045, log-rank test; FIG. 5a). However, it was also found that the above-median group had a significantly higher percentage of stage 3 and 4 tumors compared to the below median group (50.5% vs. 20%), suggesting that this difference may be the force driving the survival differences. Thus, the analysis was confined to only individuals with stage IV tumors. Using the same cut-off value as before (0.79), it was observed that individuals with scores below the cut-off tended to do better than individuals with higher scores, but the difference was not statistically significant (P=0.24). However, using a lower cut-off value (0.72), a significant difference between the groups was observed (P<0.05, FIG. 5b).

### Example 3

### Summary of Amino Acid Profiling of Examples 1 and 2

The work described above examine serum amino acid profiles in a large series of renal cell carcinoma patients and age and sex matched controls. Statistically significant differences were observed in the concentrations of 15 of the twenty-six amino acids that were quantitated. Thirteen of fifteen significantly altered of the amino acids were decreased in RCC patients relative to controls. Factor analysis indicates that a single underlying factor could account for up to 45% of the variance in amino acid levels. Without intending to be limited to any particular mechanism or theory of action, a possible explanation for this finding would be that kidney tumors might be affecting the reabsorption of amino acids by affecting overall kidney function. However, an analysis of GFR rates in the patient samples show no overall correlation between kidney function and amino acid levels, suggesting this hypothesis is incorrect. An alternative hypothesis is that the generally lower levels of serum amino acids may be a reflection of the increased usage of amino acids by tumor for biosynthetic processes. It has been proposed that weight loss in cancer patients may be responsible for this decrease in amino acid levels, but it should be noted that in this study, there was no difference in BMI between cases and controls.

A logistic regression model was identified in which a combination of eight amino acids could be used to distinguish cases from controls. ROC analysis of this model indicates that the AUC is 0.81, in a range similar to that used in other cancer screening tests such as Pap smears (0.70) and PSA tests (0.68). An important feature of the test is that it was possible to identify early stage tumors with only slightly less efficiency as late stage tumors (AUC 0.76).

The logistic regression model had prognostic utility with regards to predicting patient survival. Patients with logistic regression scores above the mean had significantly shorter survival than those with lower scores. Much of this difference appeared to be due to the fact that higher stage cancers tended to have higher regression scores. However, it was also observed that stage IV patients with the lowest regression scores survived significantly longer than patients with higher scores, indicating it may be possible to identify those stage IV patients that are most likely to benefit from aggressive therapy.

### Example 4

### Improving Predictive Power of the Model by Adding Serum Creatinine Analysis

Creatinine level determination. Creatinine levels for were determined in 277 patient serum samples (104 controls and 173 cases).

Model construction. Logistic regression was used to develop a new model containing creatinine by combining the determined creatinine level with the model score obtained for each sample using the amino acid logistic regression equation described above. The new combined model score (Mod+Cre) was then used to calculate AUROC and for survival analysis. Model building and survival analysis were performed using Statistica 10.0 software (StatSoft, Tulsa Okla.).

Results. Five additional variables were analyzed to determine if they could increase the predictive power of the model. The variables examined included serum creatinine, glucose, LDH, sodium, and calcium. In univariate analysis, only creatinine showed a significant difference between the experimental and control groups. To determine if the addition of creatinine could improve the predictive model, logistic regression was used to add the creatinine level to the existing amino acid model.

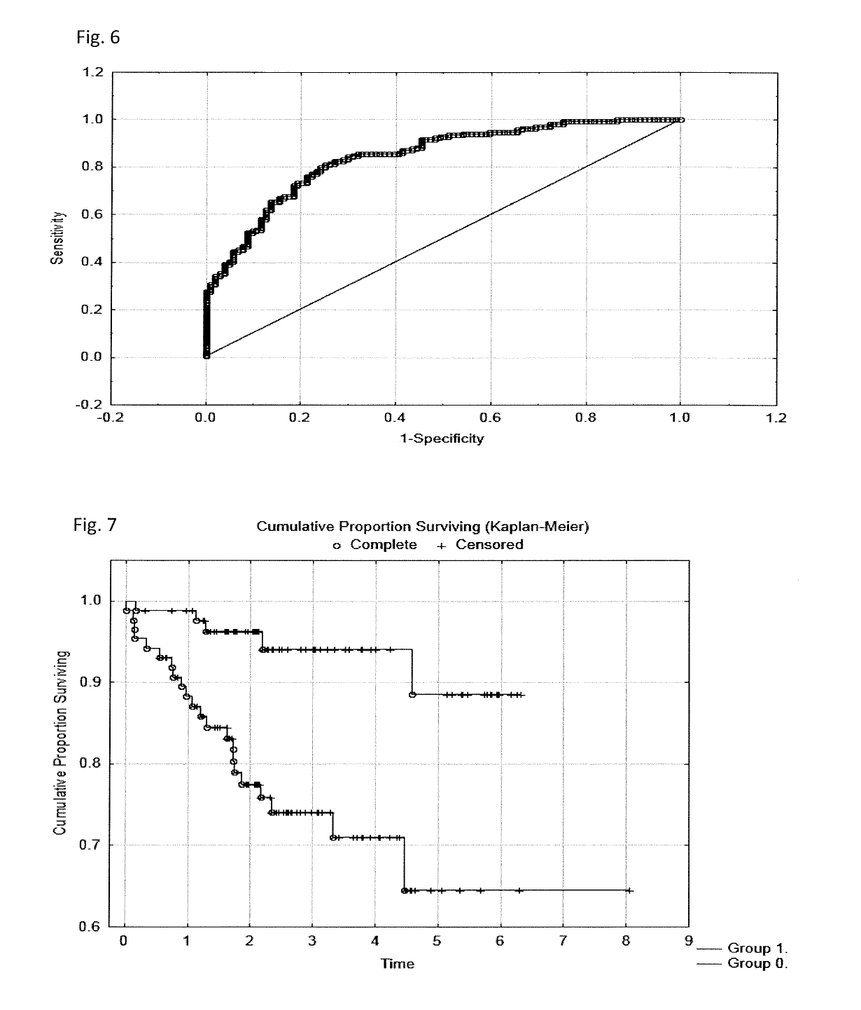

It was observed that addition of serum creatinine to the amino acid data improved the predictive power of the model. The overall AUROC increased from about 0.81 to about 0.85 when serum creatinine was combined with the regression score from the original amino acid model, the result of which was the creation of a new model (FIG. 6).

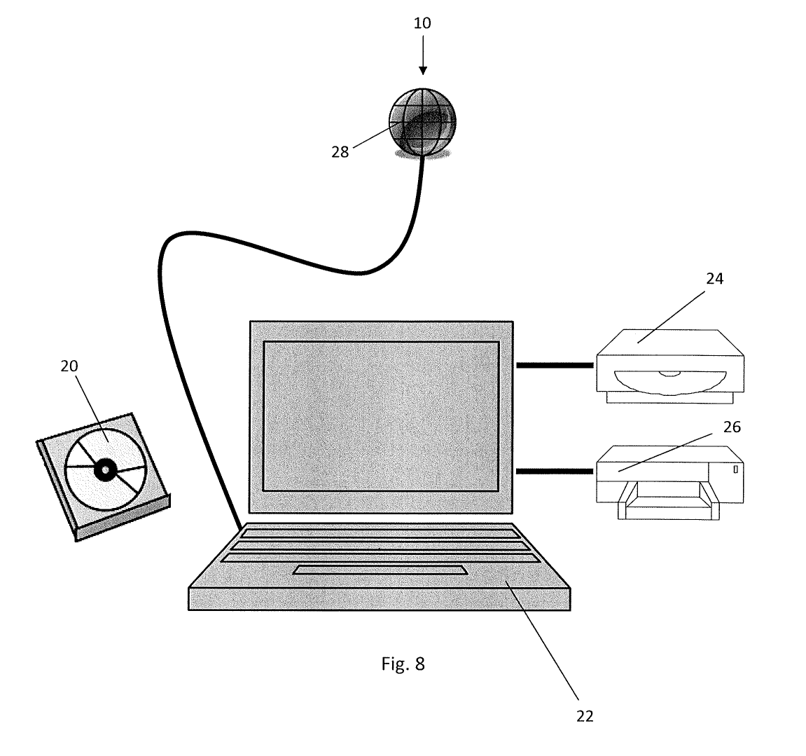

It was found that this new model also has utility for predicting overall patient survival. Patients with model scores above the mean (Group 0) showed significantly worse total overall survival compared to patients with model scores below the mean (Group 1) (FIG. 7).

### Example 5

### Confirming Metabolic Profiling as a Screen for Renal Cell Carcinoma

Fox Chase Cancer Center is a large referral facility for renal cell carcinoma by virtue of its expertise in Renal Cell Carcinoma treatment. A centralized Kidney Cancer Database has been established in which patients consent, and plasma and tumor samples are collected before surgery and stored in an in-house repository. Over 400 pieces of patient information are collected for each sample, and linked in a centralized database. This information includes complete patient demographics, disease characteristics, comorbidities, clinical laboratory data, tumor pathology, and current cancer status, including dates of recurrence and death. As of September 2011, the repository had plasma samples from over 900 RCC patients, and it continues to accrue additional samples at a rate of 150 new patients per year. In addition, the repository has started collecting longitudinal samples on a subset of patients returning for routine surveillance. The repository also has over 3,900 plasma samples from consented control, non-RCC individuals.

Complete Amino Acid Sample Preparation and Analysis. Plasma samples must first be deproteinized and subject to chemical reduction before they can be subjected to amino acid analysis. Five microliters of 12% dithiothreitol will be added to fifty microliters of plasma and samples will be incubated at room temperature for 5 minutes to reduce the samples. Next, to deproteinate the samples, 55 microL of 10% sulfosalicylic acid will be added, and the samples will then be incubated for one hour at 4° C. Samples will then be centrifuged at 12,000×g for ten minutes, and the supernatant will be collected and loaded into auto-loading tubes. Auto-loading tubes will then be fed into a BioChrom® 30 amino acid analyzer, and peaks will be identified and quantitated using EZ Lite software.

Quantitation of the different amine containing compounds will be determined by comparing peak area to a known standard. Groups of 12-16 samples containing alternating control patient and cancer patient samples will be run together along with a quantitation standard. Since it takes approximately three hours for the machine to analyze each sample, groups of this size will take about two days of instrument time per run.

Sample Size Considerations. For these experiments, it is anticipated that at least 200 RCC patient samples and 200 control samples will be used. Table 5 presents the detectable odds ratios in multiple logistic regressions with 200 cases and 200 controls. Estimates are presented over a range of assumptions about the probability of being a control when all amino acids are at their means. Estimates are also presented over a range of assumptions about the squared coefficient of multiple correlation (R2) that measures the association of an amino acid of interest with other amino acids entered as covariates in a regression model. The R2 value can be obtained by fitting a linear regression model of an amino acid's expression levels with the other amino acid levels as covariates.

TABLE 5
Detectable Odds Ratios in Multiple Logistic Regressions

| | R₂ when an amino | Probability of being | |
|---|---|---|---|
| Power assuming | acid of interest is | a control at the | |
| 1% Type I error | regressed on other | mean amino acid | Detectable |
| rate (2--sided) | covariates | covariate level | odds ratio |
| 85% | 0 | 30% | 1.48 |
| 85% | 0 | 50% | 1.44 |
| 85% | 0 | 70% | 1.48 |
| 85% | 0.3 | 30% | 1.60 |
| 85% | 0.3 | 50% | 1.54 |
| 85% | 0.3 | 70% | 1.60 |
| 85% | 0.5 | 30% | 1.75 |
| 85% | 0.5 | 50% | 1.67 |
| 85% | 0.5 | 70% | 1.75 |

The detectable odds ratio is the odds ratio associated with a one standard deviation increase in an amino acid covariate level. Table 5 shows sufficient power to detect modest associations under all of the assumptions, with a modest association including one in which the odds ratio is less than 2.0. Type I error rates of 1% (2-sided) are assumed.

Data Analysis. The data set generated from the amino acid analysis will be quite substantial. For each patient and control, the data will include the 26 amino acids, sex, BMI, age, and race (31 variables). For the patients, additional data will include tumor type (i.e., clear cell, papillary, etc.), size, clinical stage, and pathologic stage. As the database is constantly being updated, additional information such as recurrence, follow-up treatment, and overall survival will be available over time.

Data exploration will be performed using Statistica 9.1 software. Initial analysis will focus on univariate analysis of each amino acid. First, it will be determined whether the amino acids concentrations are normally distributed and any variables will be logged if required. The means of cases and controls for each amino acid will be compared using a two-sided t-test, or non-parametric test if appropriate. It will be determined if there are differences in each amino acid associated with clinical stage of the tumor (e.g., is the serum profile of patients with stage 1 patients different than stage 4 patients). For multiple group analysis, ANOVA will be used.

Preliminary data indicated that the serum amino acid levels tend to be correlated with each other. The mean (±SD) correlation for all the amino acids with each other is R=0.44 (±0.22). The mean for each amino acid in a model in which each mean is adjusted for all the other amino acids at their mean will also be determined using the generalized linear modeling module in Statistica software.

To determine if amino acid analysis can effectively identify cases from controls, a logistic regression procedure will be used. Variables that have been identified as being significantly different between cases and controls will first be put into a logistic regression model using forward step-wise regression to select the most powerful predictors. At each step, the least predictive variable was removed based on the Wald score. The final model contained only those variables with Wald scores with P<0.05.

Constructing receiver-operator curves and conducting AUC analysis will examine the robustness of the model. To guard against potential model over fitting, a 10-fold cross-validation analysis will be performed on each model. If cross-validation reveals evidence of over-fitting, the number of variables in the model will be reduced. Classification and Regression Trees (CART) methods will also be used to explore the relationship between the amino acids and case/control status. Unlike the standard CART approach where there is no concept of statistical significance in the algorithm, a unified framework proposed by that embeds recursive binary partitioning into the theory of permutation tests will be used (Hothorn T et al. (2006) J. Computational and Graphical Statistics 15:651-74). Each classification method has its own particular strengths and weaknesses, so it is important to try a variety of methods to obtain the best model. Both of these options are integrated in the Statistica software package.

Discussion of Specificity and Sensitivity Issues. Preliminary data show that 38.1% sensitivity with 3.8% false positives can be achieved. The following Examples will discuss two strategies to find additional metabolomic markers that might be used to improve the test.

Addition of Other Serum Clinical Markers to the Model. Patients undergoing surgery for RCC are each given a Chem 14 metabolic panel. The data collected from this panel include sodium, potassium, chloride, bicarbonate, calcium, ionized calcium, urea nitrogen, creatinine (from which eGFR can be calculated), glucose, total protein, albumin, globulin, bilirubin, Aspartate aminotransferase (AST), and Alanine amino transferase (ALT). This same test will be performed on serum from control subjects, and will determine whether any of these metabolites vary significantly between RCC patients and controls. Metabolites that vary will be included in the logistic regression model, and whether they can increase the specificity or sensitivity of the test using ROC analysis will be determined.

Creatinine levels in controls were significantly lower than in RCC patients (0.82 mg/dl controls vs. 1.07 mg/dl patients P<0.000012). When creatinine was added to the logistic regression model, the area under the ROC increased (FIG. 6). This model achieved 43.3% sensitivity with only 2.9% false positives.

Metabolomic Studies. An analytical platform will be used to conduct comprehensive metabolomic analyses. The system will incorporate two separate ultrahigh performance liquid chromatography/tandem mass spectrometry injections that can quantitate 264 small metabolites in human serum (Evans A M et al. (2009) Anal. Chem. 81:6656-67). One hundred control and 100 age-matched RCC patient samples will be analyzed according to this platform to determine metabolites that are differentially expressed at statistically significant levels between cases and controls. Once all changed metabolites have been identified, those metabolites having the highest discriminatory power will become the primary focus, with the expectation that such may include metabolites for which clinical tests are already routinely performed. A subset of these markers will be selected and combines with amino acid analysis (done on the same group of samples). Logistic regression methodology will then be used to create a model to distinguish cases and controls. To confirm the validity of this model, these findings will be tested on an independent set of 200 patients and controls.

### Example 6

### Evaluation of Amino Acid Profiling in Identifying Recurrence of RCC

Preliminary Data. In order for amino acid profiling to be useful in detecting recurrence, the assay needs to have relatively low amounts of intra-individual variation. Previously, it has been reported that the intra-individual variability in amino acid levels is significantly less than the inter-individual variability (Scriver G R et al. (1985) Metabolism 34:868-73). To confirm this finding, a pilot study was carried out in which intra-individual variability of amino acid levels in a group of 20 individuals was determined by drawing blood at two different time points. The mean intra-individual CV for all the amino acids was 16%, while the mean inter-individual CV for all 26 amino acids in 104 controls was 33%. These data support the idea that amino acid profiles are significantly more stable within an individual than among individuals.

Sample Acquisition. As described in Example 5, samples will be obtained from the in house repository. The Repository has recently started collecting “longitudinal” samples from RCC patients when they return for routine monitoring after surgery. Patients with high risk of recurrence, e.g., stage III or stage IV patients with undetectable disease by CT after surgery will be the focus of additional investigations. Recurrence, as detected by routine scanning, is recorded in a database, and this information will be collected for each patient.

Data Analysis. Data for 26 different amino acids will be collected at six different time points from 100 patients. Data will be analyzed at several different levels. First, whether amino profiles change as a result of surgery will be assessed. This will be possible because the first collection will occur before surgery has occurred. Each amino acid will be analyzed separately, and also together, using the logistic regression model score developed in the preliminary data from the foregoing Examples. It is expected that immediately following surgery, the model score will adjust downward toward a more normal value. If this is not the case, a new logistic regression analysis will be performed to identify changes that are the best predictors, presurgery vs. post-surgery. Next, the model will be used to evaluate each sample at each time point and to determine whether changes in the model score are associated with tumor recurrence in the sample set.

To investigate time trends in the association of the amino acid profiles with recurrence, time until recurrence in which amino acids are entered or their summary scores as covariates will be fit into Cox proportional hazards regressions. It will include change scores between measurement times as time dependent covariates in the models to investigate how changes in amino acid levels are associated with recurrence. It is not expected that death from other causes before recurrence will be a significant competing risk in this study. However, in the unlikely event that many people die from other causes prior to recurrence, the Fine and Gray proportional hazards regressions model will be used to account for the competing risk of death.

### Example 7

### Determining if Alterations in Serum Amino Acids are Unique to RCC

Sample Acquisition and Processing. Samples will be obtained from the in-house repository. As of September 2011, the repository had blood and serum from 1032 lung cancer patients, 2330 breast cancer patients, 1878 prostate cancer patients, and 527 colon cancer patients. All serum samples were taken prior to surgery. Information about each sample includes sex, age, stage, grade, and tumor size. Two hundred samples of each tumor type will be selected for analysis. A control group for each tumor type will be created by matching each sample with control individuals on the basis of sex and age. Serum will be processed and analyzed using the Biochrom® 30 amino acid analyzer.

Data Analysis. The data set generated from the amino acid analysis will be quite substantial. Each patient and control group will include data on 26 amino acids, sex, age, tumor stage, tumor size and tumor grade. Data will be collected and handled as described in Example 5 for the RCC patients. Univariate analysis of each amino acid will be performed, and the means will be compared to case and control group for each cancer using a two-sided t-test, or non-parametric test if appropriate. Whether there are differences in each amino acid associated with clinical stage of the tumor (e.g., is the serum profile of patients with stage 1 patients different than stage 4 patients) will also be evaluated. For multiple group analysis, ANOVA will be used.

In preliminary experiments, it was observed that the serum amino acid levels tend to be correlated with each other. The mean (±SD) correlation for all the amino acids with each other is R=0.44 (±0.22). The mean for each amino acid will be determined using a model in which each mean is adjusted for all the other amino acids at their mean, using the generalized linear modeling module in Statistica.

To determine if amino acid analysis can effectively identify cases from controls, a logistic regression procedure will be used. Variables that have been identified as being significantly different between cases and controls will first be put into a logistic regression model using forward step-wise regression to select the most powerful predictors. Constructing receiver-operator curves and conducting AUC analysis will examine the robustness of the model. To guard against potential model over-fitting, 10-fold cross-validation analyses will be performed on each model. If cross-validation reveals evidence of over-fitting, the number of variables in the model will be reduced.

Classification and Regression Trees (CART) methods will also be used to explore the relationship between the amino acids and case/control status. Unlike the standard CART approach where there is no concept of statistical significance in the algorithm, the unified framework proposed by Hothorn et al. that embeds recursive binary partitioning into the theory of permutation tests will be used (Hothorn T et al. (2006) J. Computational and Graphical Statistics 15:651-74). Each classification method has its own particular strengths and weaknesses, so it is helpful to try a variety of methods to obtain the best model. Both of these options are integrated in the Statistica software package.

If it is observed that amino acid profiles are predictive of cancers other than RCC, the nature of the predicative profile will be explored using similar methodologies. Whether the amino acids themselves and the direction of the changes are similar to those observed in the RCC samples will be evaluated. If there are differences, the extent to which the different models specify the type of cancer will be examined. A multinomial logit model will be created for this purpose. These models are similar to logistic regression, but can be used to classify multiple categorically distributed dependent variables.

The invention is not limited to the embodiments described and exemplified above, but is capable of variation and modification within the scope of the appended claims.

1-27. (canceled)

28. A system for diagnosing renal cell carcinoma, comprising a data structure comprising one or more reference profiles comprising a reference concentration for each amino acid in a first panel of amino acids comprising alanine, asparagine, arginine, citrulline, cysteine, glutamate, glycine, histidine, methionine, phenylalanine, proline, serine, taurine, threonine, and tyrosine, or a second panel of amino acids comprising cysteine, histidine, leucine, lysine, ornithine, proline, tyrosine, and valine, and optionally a reference concentration for creatinine, and a processor operably connected to the data structure, wherein the reference profiles include one or more of a reference profile for a healthy subject, a reference profile for a subject at risk for developing renal cell carcinoma, a reference profile for a subject at risk for developing recurrent renal cell carcinoma, and a reference profile for a subject having renal cell carcinoma, and wherein the processor is programmed to compare the concentration of each amino acid in the first panel of amino acids determined from a sample of serum obtained from a subject with the reference concentration for each amino acid in the first panel in the one or more reference profiles, to compare the concentration of each amino acid in the second panel of amino acids determined from a sample of serum obtained from a subject with the reference concentration for each amino acid in the second panel in the one or more reference profiles, and to compare the concentration of creatinine determined from a sample of serum obtained from a subject with the reference concentration for creatinine in the one or more reference profiles.

29. The system of claim 28, wherein the reference profile comprises a reference concentration for each amino acid in the first panel, and the reference concentration for each of alanine, asparagine, citrulline, glutamate, glycine, histidine, methionine, phenylalanine, proline, serine, taurine, threonine, and tyrosine is lower than in the serum concentration of alanine, asparagine, citrulline, glutamate, glycine, histidine, methionine, phenylalanine, proline, serine, taurine, threonine, and tyrosine in a healthy subject, and the reference concentration for arginine and cysteine is higher than the serum concentration of arginine and cysteine in a healthy subject.

30. The system of claim 28, wherein the reference profile comprises a reference concentration for each amino acid in the second panel, and the reference concentration for each of histidine, leucine, lysine, ornithine, proline, tyrosine, and valine is lower than in the serum concentration of histidine, leucine, lysine, ornithine, proline, tyrosine, and valine and the reference concentration for cysteine is higher than the serum concentration of cysteine in a healthy subject.

31. The system of claim 28, wherein the reference profile for a subject having renal cell carcinoma comprises one or more of a reference profile for a subject having stage I renal cell carcinoma, a reference profile for a subject having stage II renal cell carcinoma, a reference profile for a subject having stage III renal cell carcinoma, or a reference profile for a subject having stage IV renal cell carcinoma.

32. The system of claim 28, wherein the processor is a computer processor.

33-36. (canceled)

37. The system of claim 28, further comprising an output for providing results of the comparison to a user.

38. (canceled)

39. The system of claim 28, further comprising executable code for causing the processor to determine a prognosis of a subject having renal cell carcinoma based on a comparison of the concentration of each amino acid in the first panel of amino acids determined from a sample of serum obtained from the subject with the reference concentration of each amino acid in the first panel of amino acids in a reference profile for a subject having renal cell carcinoma.

40. The system of claim 28, further comprising executable code for causing the processor to determine a prognosis of a subject having renal cell carcinoma based on a comparison of the concentration of each amino acid in the first panel of amino acids and the concentration of creatinine determined from a sample of serum obtained from the subject with the reference concentration of each amino acid in the first panel of amino acids and the reference concentration of creatinine in a reference profile for a subject having renal cell carcinoma.

41-46. (canceled)

47. The system of claim 28, further comprising a computer network connection.

48-65. (canceled)

66. The system of claim 28, further comprising executable code for causing the processor to determine a prognosis of a subject having renal cell carcinoma based on a comparison of the concentration of each amino acid in the second panel of amino acids determined from a sample of serum obtained from the subject with the reference concentration of each amino acid in the second panel of amino acids in a reference profile for a subject having renal cell carcinoma.

67. The system of claim 28, further comprising executable code for causing the processor to determine a prognosis of a subject having renal cell carcinoma based on a comparison of the concentration of each amino acid in the second panel of amino acids and the concentration of creatinine determined from a sample of serum obtained from the subject with reference concentration of each amino acid in the second panel of amino acids and the reference concentration of creatinine in a reference profile for a subject having renal cell carcinoma.

68. The system of claim 39, wherein the prognosis comprises a substantial likelihood of mortality within about five years.

69. The system of claim 40, wherein the prognosis comprises a substantial likelihood of mortality within about five years.

70. The system of claim 66, wherein the prognosis comprises a substantial likelihood of mortality within about five years.

71. The system of claim 67, wherein the prognosis comprises a substantial likelihood of mortality within about five years.

72. The system of claim 28, wherein the subject is a human being.

73. A method for diagnosing renal cell carcinoma, comprising:
(a) determining the concentration of each amino acid in a panel of amino acids comprising alanine, asparagine, arginine, citrulline, cysteine, glutamate, glycine, histidine, methionine, phenylalanine, proline, serine, taurine, threonine, and tyrosine, and optionally determining the concentration of creatinine, in a sample of serum obtained from a subject;
(b) entering the determined concentration of each amino acid in the panel, and if the concentration of creatinine was determined, entering the determined concentration of creatinine into the system of claim 28;
(c) causing the processor of the system to compare the entered determined concentration of each amino acid from step (b) with the reference concentration for each amino acid in the first panel in one or more reference profiles, and if the determined concentration of creatinine was entered, causing the processor of the system to compare the entered determined concentration of creatinine from step (b) with the reference concentration for creatinine in the one or more reference profiles; and
(d) determining whether the subject is healthy, is at risk for developing renal cell carcinoma, is at risk for developing recurrent renal cell carcinoma, or has renal cell carcinoma based on the comparison from step (c).

74. A method for diagnosing renal cell carcinoma, comprising:
(a) determining the concentration of each amino acid in a panel of amino acids comprising cysteine, histidine, leucine, lysine, ornithine, proline, tyrosine, and valine, and optionally determining the concentration of creatinine, in a sample of serum obtained from a subject;
(b) entering the determined concentration of each amino acid in the panel, and if the concentration of creatinine was determined, entering the determined concentration of creatinine into the system of claim 28;
(c) causing the processor of the system to compare the entered determined concentration of each amino acid from step (b) with the reference concentration for each amino acid in the second panel in one or more reference profiles, and if the determined concentration of creatinine was entered, causing the processor of the system to compare the entered determined concentration of creatinine from step (b) with the reference concentration for creatinine in the one or more reference profiles; and
(d) determining whether the subject is healthy, is at risk for developing renal cell carcinoma, is at risk for developing recurrent renal cell carcinoma, or has renal cell carcinoma based on the comparison from step (c).
